(19) United States
(12) Patent Application Publication
(10) Pub. No.: US 2012/0021048 A1
(43) Pub. Date: Jan. 26, 2012

(54) Combination Dosage Compositions Comprising a Cholesterol-Lowering Agent, a Renin-Angiotensin System Inhibitor, an Antioxidant Agent and an Antiplatelet Agent for Treatment and Prevention of Cardiovascular Disease

(75) Inventors: Miguel Angel Duarte-Vazquez, Irapuato (MX); Sandra Garcia Padilla, Tula de Allende (MX); Jorge L. Rosado, Queretaro (MX)

(73) Assignee: Nucitec S.A. de C.V., Colonia Penuelas (MX)

(21) Appl. No.: 13/188,260

(22) Filed: Jul. 21, 2011

Related U.S. Application Data
(60) Provisional application No. 61/366,398, filed on Jul. 21, 2010.

(51) Int. Cl. A61K 31/60; A61K 9/48; A61K 31/01; A61P 9/00; A61K 31/40; A61K 31/403

(52) U.S. Cl. 424/451; 514/423; 514/412; 514/161; 514/762

(57) ABSTRACT

The present invention is in the fields of medicine, pharmaceuticals, nutraceuticals and cardiology. The invention provides compositions comprising a statin, an inhibitor of the angiotensin converting enzyme, an antiplatelet compound and an antioxidant compound for the treatment and/or prevention of cardiovascular disease.

### BACKGROUND OF THE INVENTION

1. Field of the Invention

The present invention is in the fields of medicine, pharmaceuticals, nutraceuticals and cardiology. The invention provides compositions comprising a statin, an inhibitor of the angiotensin converting enzyme, an antiplatelet compound and an antioxidant compound for the treatment and/or prevention of cardiovascular disease.

2. Related Art

Cardiovascular Disease

Cardiovascular disease (CVD) is a major public health concern. CVD is the general term for heart and blood vessel diseases, including atherosclerosis, coronary heart disease, cerebrovascular disease, aorto-iliac disease, and peripheral vascular disease. Patients suffering from manifestations of cardiovascular disease may develop a number of complications, including but not limited to myocardial infarction, stroke, angina pectoris, transient ischemic attacks, congestive heart failure, aortic aneurysm, and death. CVD is the leading cause of death in the world (World Health Organization, Cardiovascular Diseases Fact Sheet, 2007: No. 317). Thus, prevention and treatment of cardiovascular disease are areas of major public health importance.

Risk factors for CVD include hypercholesterolemia, systemic hypertension, smoking, diabetes, hyperhomocysteinemia, visceral obesity and the aging process. All of these risk factors have been associated with impairment of vascular endothelium function and promotion of atherosclerotic plaque formation in patients with CVD (Naghavi M. et al., Circul., 108:1664-1672, 2003). Impaired vascular endothelium may promote inflammation, oxidation of lipoproteins, smooth muscle proliferation, extracellular matrix deposition or lysis, accumulation of lipid-rich material, platelet activation and thrombus formation. All of these consequences may contribute to the development and clinical expression of atherosclerosis. Thus, abnormalities in the proper functioning of vascular endothelium likely contribute to the pathogenesis of CVD.

Hypercholesterolemia, LDL oxidation and angiotensin II degradation have been identified as some of the most important promoters of the vascular endothelial damage. LDL cholesterol is easily oxidized and in this form displays a high affinity for cell (particularly endothelial cell) surfaces, leading to formation of foam cells loaded with cholesterol esters. These foam cells are accompanied by proliferation of smooth muscle cells in the vasculature and elaboration of an extracellular lipid core and fibrous cap. Thus, patients exhibiting hypercholesterolemia are at high risk for the development and progression of CVD.

Hypercholesterolemia is also an established risk factor for CVD, and the close relation between hypercholesterolemia and atherosclerosis is well-known. Endothelium-dependent vascular relaxation is impaired in hypercholesterolemic patients regardless of whether or not other coronary risk factors exist. Endothelial dysfunction and impairment arises quickly in hypercholesterolemic patients.

An increase in the concentration of the low density lipoprotein (LDL) fraction in the peripheral and coronary circulation has been identified as one of the main contributors to CVD. LDL induces upregulation of the expression of angiotensin II type 1 receptor in isolated vascular smooth muscle cells. Overexpression of angiotensin II type 1 receptors may account for enhanced release of free radicals and increased vasoconstriction and cell proliferation, which also appear to contribute to the onset of CVD.

Angiotensin II is a potent vasoactive peptide that produces acute systemic and local vasoconstriction resulting in high blood pressure (hypertension). Angiotensin II also contributes to the long term progression of the atherosclerotic process. Recent studies have shown that LDL induces the expression of angiotensin II type I receptors, thus increasing blood pressure. Angiotensin II has been reported to accelerate the process of atherosclerosis through anion generation and promotion of endothelial dysfunction (Fukai T. et al., Circ. Res. 85:23-28, 1999). Further studies have shown that simvastatin, a statin that lowers LDL levels, reversed the elevated blood pressure induced by angiotensin II infusion in rabbits (Nickening G. et al., Circul. 100:2131-2134, 1999). Thus, angiotensin II contributes to the onset of cardiovascular disease.

Experimental and epidemiological data suggest that activation of the rennin-angiotensin-aldosterone system and oxidative modification of LDL-cholesterol play important roles in pathogenesis (Lonn E M et al., Circul. 90:2056-2069, 1994). Angiotensin II exerts multiple effects on the cardiovascular system including hypertension and cardiovascular hypertrophy, and free radical production has been proposed as a mechanism participating in angiotensin II-induced cardiovascular alterations. Through stimulation of its type I receptor, angiotensin II induces over-expression of cytosolic proteins involved in the activation of the NADPH oxidase of vascular endothelial cells, smooth muscle cells and leukocytes. In these cells, angiotensin II favors the production of reactive oxygen species (ROS) such as superoxide anions, hydrogen peroxide, and hydroxyl radicals. Together with leukocyte adhesion and proliferation and migration of various cell types, these events may lead to phenotype transformation of the arterial wall and vascular hypertrophy, all of which can lead to CVD.

Inhibition of the production of superoxide anions thus limits the oxidation of LDL, and contributes to an increase in nitric oxide (NO) bioactivity by enhancing NO synthesis and limiting oxidative degradation of NO. The angiotensin converting enzyme (ACE) inhibitor enalapril, has been shown to reduce intracellular production of superoxide anions thus protecting LDL cholesterol from oxidation and improving endothelial function (Koh K K et al., Am. J. Cardiol. 83:327-331, 1999).

An increase in the concentration of the low density lipoprotein (LDL) fraction in the peripheral circulation as well as the oxidation of such lipoproteins, play a key role in the pathogenesis of the atherosclerosis and CVD, therefore an intervention which reduces LDL synthesis and its oxidation could represent a powerful tool in the management of CVD. Recently, growing evidence from several epidemiological studies has indicated that lycopene plays an important role in the primary prevention of CVD (Rissanen T H et al., Am. J. Clin. Nutr. 77:133-138, 2003; Sesso H D et al., Am. J. Clin. Nutr. 79:47-53, 2004). Lycopene is a highly unsaturated acyclic carotenoid found predominantly in tomato products. This lipophilic compound is transported in the blood by lipoproteins and accumulates in many human tissues including the vasculature.

Lycopene is a potent chemical antioxidant shown to quench oxygen species in vitro (Bohm F. et al., Nat. Med. 1:98-99, 1995) and to decrease blood lipid biomarkers of oxidative stress in vivo (Rao A V and Awarwall S. Nutr. Cancer. 31:199-203, 1998). Cell cultures experiments have established that lycopene can inhibit the destruction of nitric oxide by superoxide anion (Panasenko O M et al., Arch. Biochem. Biophys. 373:302-305, 2000) and attenuate inflammatory cytokine-stimulated endothelial cell adhesion molecule expression and monocyte-endothelial interactions (Martin K R, et al. Atherosclerosis. 150:256-274, 2000). A number of previous studies have reported that increase dietary consumption of lycopene reduce oxidative stress and oxidation of lipids (Visioli F. et al., Eur. J. Clin. Nutr. 42:201-206, 2003; Porrini M. et al., Br. J. Nutr. 93:93-99, 2005; Riso P. et al., J. Agric. Food. Chem. 54:2563-2566, 2006).

Previous work has also shown that lycopene can reduce LDL cholesterol levels. In cell cultures, the addition of lycopene to macrophages suppresses cellular cholesterol synthesis by approximately 40% by directly inhibiting cellular HMG CoA (hydroxy-3-methyl-glutaryl-Coenzyme A) activity (Fuhrman B. et al., Biochem. Biophys. Res. Commun. 233:658-662, 1997). This suppression was concomitant with an increased uptake and degradation of LDL particles by macrophages, suggesting simultaneous up-regulation of LDL-receptor activity. The same authors tested in vivo pure lycopene supplementation, and showed that lycopene reduces plasma LDL cholesterol concentration by 14%.

Lycopene may reduce carotid intima-media thickness and lowers incidence of myocardial infarction, ischemic heart disease, and stroke mainly by reducing LDL cholesterol concentrations and inducing the ability of LDL particles to resist ion-induced oxidation.

Furthermore, inhibition of platelet activation at sites of vascular injury has other indirect actions such as reducing the release of inflammatory cytokines which also has an impact on attenuating major vascular events. Recent studies have indicated that treatment with a low dose of the antiplatelet agent acetylsalicylic acid reduces the activation and aggregation of platelets by inhibiting cyclooxygenases (COX-1 and COX-2) and the formation of thromboxane (Patrono C. et al., N. Engl. J. Med. 353: 2373-2383, 2005). Additionally, it has been shown that statin treatment, such as with simvastatin, also reduces platelet aggregation, possibly via reduction of thromboxane A2 production and cholesterol content of platelet membranes, and reduces thrombogenic potential, via an effect on tissue factor (Ferro D, et al., Atherosclerosis 149:111-116, 2000).

McGovern et al., in U.S. Pat. No. 5,140,012, disclose the use of pravastatin alone or in combination with an angiotensin converting enzyme (ACE) inhibitor, to prevent onset of restenosis following angioplasty. The disclosure is limited to a single inhibitor of HMG CoA, pravastatin.

U.S. Pat. Nos. 5,461,039 and 5,593,971 disclose the use of a cholesterol-lowering drug, alone or in combination with ACE inhibitors, to reduce hypertension in a normotensive individual who has insulin resistance. The disclosed methods are limited to use in normotensive individuals who are insulin-resistant.

Olokutan et al., in U.S. Pat. No. 5,622,985, disclose that HMG CoA inhibitors, particularly pravastatin, when used alone or alternatively in combination with an angiotensin converting enzyme (ACE) inhibitor, decrease the risk of a second heart attack in a patient who has a substantially normal cholesterol level.

Mexican Patent No. MX218975 entitled “Composición Farmacéutica que Contiene Estatina y Aspirina” discloses the use of a statin in combination with acetylsalicylic acid to reduce hypercholesterolemia and the risk of myocardial infarction.

Alvarez-Ochoa et al., in Mexican request patent number PA/A/2005/014063, disclose the use of a pharmaceutical composition comprising an antihypertensive and a cholesterol lowering compound. The selected hypolipidemic agent and antihypertensive are simvastatin and amlodipine, respectively. The disclosure is limited to the use of amlodipine as the antihypertensive agent.

Currently, however, there does not exist a treatment or preventative for cardiovascular disease that addresses more than one underlying cause of CVD; there are only certain treatment strategies that are frequently used to treat, or to control and diminish, the individual symptoms or underlying risk factors of CVD, such as those treatments directed at hypercholesterolemia and hypertension individually.

Pharmacological Treatment of Cardiovascular Disease Symptoms

There has been an ongoing search for methods of effective treatment and/or prevention of CVD, as well as for long term treatment thereof, including methods that would treat and/or prevent the underlying risk factors for CVD such as hypertension and hypercholesterolemia. Treatments for cardiovascular diseases and the associated risk factors include, for example, administration of vasodilators, angioplasty and bypass surgery. Such treatments have met with disapproval due to the increased risks versus the marginal or temporary benefits gained by such treatments. Such treatments have serious shortcomings in long term effectiveness. The use of vasodilator drugs and mechanical treatments for acute and chronic occlusive vascular diseases of the heart, and of the central and peripheral vascular system, have to date been ineffective for obtaining favorable long term results. The outcome with pharmaceutical treatments is also minimally effective because the treatments are typically directed toward the effects of individual underlying factors, but do not address the causes of CVD in a concerted manner, e.g., treating the several factors associated with the disease including hypertension, hypercholesterolemia, lipids oxidation, platelet aggregation and inflammation.

Pharmacological treatment of the disease symptoms of CVD generally includes a variety of approaches focused on controlling and diminishing the individual underlying causes associated with the disease. Previously reported pharmaceutical approaches to treat CVD include using known active ingredients, such as vasodilators, angiotensin II receptor antagonists, angiotensin converting enzyme inhibitors, diuretics, antithrombolytic agents, β-adrenergic receptor antagonists, α-adrenergic receptor antagonists, calcium channel blockers and the like.

There is a need in the art for a specific approach to treating and/or preventing cardiovascular disease, which not only improves and alleviates the symptoms associated with CVD but which also affects and simultaneously reverses the different underlying physiological causes of the disease.

### BRIEF SUMMARY OF THE INVENTION

The present invention provides a pharmaceutical composition for the treatment of cardiovascular disease in mammals, comprising a therapeutically effective amount of each of at least one cholesterol lowering agent (e.g., atorvastatin, simvastatin, or rosuvastatin), at least one angiotensin converting enzyme inhibitor (e.g., ramipril or enalapril), at least one antiplatelet agent (e.g., acetylsalicylic acid) and at least one antioxidant agent (e.g. lycopene, Vitamin E, Vitamin C or beta-carotene). In certain embodiments, the cardiovascular disease is hypercholesterolemia or hypertension. In some embodiments, the invention provides for a pharmaceutical composition comprising ramipril, atorvastatin, lycopene and an antiplatelet agent. In further embodiments, the invention provides a pharmaceutical composition comprising ramipril, atorvastatin, lycopene and acetylsalicylic acid. In other embodiments, the invention provides a pharmaceutical composition comprising from about 1 mg to about 80 mg, from about 5 mg to about 60 mg, or about 5 mg of ramipril. In some embodiments, the invention provides a pharmaceutical composition(s) comprising from about 5 mg to about 140 mg, from about 20 mg to about 80 mg, or about 20 mg of atorvastatin. In certain embodiments, the invention provides a pharmaceutical composition(s) comprising from about 20 mg to about 500 mg, from about 35 mg to about 350 mg, from about 35 to about 100 mg or about 81 mg of acetylsalicylic acid. In yet other embodiments, the invention provides a pharmaceutical composition(s) comprising from about 5 mg to about 500 mg, from about 30 mg to about 300 mg, from about 30 to about 100 mg or about 60 mg of lycopene. The compositions can be in either aqueous solution or solid form and can be administered orally (e.g., capsule, tablet, or powder), parenterally or topically as a single dose that is preferably administered once daily.

Other features and advantages of the invention will be apparent from the following detailed description, and from the claims. The disclosed materials, methods, and examples are for illustrative purposes only and are not intended to be limiting. Skilled artisans will appreciate that methods and materials similar or equivalent to those described herein can be used to practice the invention.

Unless otherwise defined, all technical and scientific terms used herein have the meaning commonly understood by one skilled in the art to which this invention belongs. All publications, patent applications, patents, and other references mentioned herein are incorporated by reference in their entirety. In case of conflict, the present specification, including definitions, will control.

### DETAILED DESCRIPTION OF THE INVENTION

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are described hereinafter.

### DEFINITIONS

About: As used herein when referring to any numerical value, the term “about” means a value of ±10% of the stated value (e.g., “about 50° C.” encompasses a range of temperatures from 45° C. to 55° C., inclusive; similarly, “about 100 mM” encompasses a range of concentrations from 90 mM to 110 mM, inclusive).

Disease, disorder, condition: As used herein, the terms “disease” or “disorder” refer to any adverse condition of a human or animal including tumors, cancer, allergies, addiction, autoimmunity, infection, poisoning or impairment of optimal mental or bodily function. “Conditions” as used herein includes diseases and disorders but also refers to physiologic states. For example, fertility is a physiologic state but not a disease or disorder; hence, compositions suitable for preventing pregnancy by decreasing fertility would therefore be described herein as a treatment of a condition (fertility), but not a treatment of a disorder or disease. Other conditions encompassed by the use of that term herein will be understood by those of ordinary skill, in the art.

Effective Amount: As used herein, the term “effective amount” refers to an amount of a given compound or composition that is necessary or sufficient to realize a desired biologic effect. An effective amount of a given compound or composition in accordance with the methods of the present invention would be the amount that achieves this selected result, and such an amount can be determined as a matter of routine by a person skilled in the art, using assays that are known in the art and/or that are described herein, without the need for undue experimentation. For example, an effective amount for treating or preventing cardiovascular disease could be that amount necessary to prevent the development and/or progression of the symptoms and/or underlying physiological causes of cardiovascular disease, such as hypercholesterolemia and hypertension. The term is also synonymous with “sufficient amount” and “therapeutically effective amount.” The effective amount for any particular application can vary depending on such factors as the disease, disorder or condition being treated, the particular composition being administered, the route of administration, the size of the subject, and/or the severity of the disease or condition. One of ordinary skill in the art can determine empirically the effective amount of a particular compound or composition of the present invention, in accordance with the guidance provided herein, without necessitating undue experimentation.

One, a, or an: When the terms “one,” “a,” or “an” are used in this disclosure, they mean “at least one” or “one or more,” unless otherwise indicated. As such, the terms “a” (or “an”), “one or more,” and “at least one” can be used interchangeably herein.

Treatment: As used herein, the terms “treatment,” “treat,” “treated” or “treating” refer to prophylaxis and/or therapy, particularly wherein the object is to prevent or slow down (lessen) an undesired physiological change or disorder, such as the development and/or progression of cardiovascular disease. Beneficial or desired clinical results include, but are not limited to, alleviation of symptoms, diminishment of the extent of disease, stabilized (i.e., not worsening) state of disease, delay or slowing of disease progression, amelioration or palliation of the disease state, and remission (whether partial or total), whether detectable or undetectable. “Treatment” can also mean prolonging survival and/or increased quality of life as compared to expected survival and/or quality of life if not receiving treatment. Those in need of treatment include those already with the condition or disorder (e.g., cardiovascular disease) as well as those prone to have the condition or disorder or those in which the condition or disorder is to be prevented. By “subject” or “individual” or “animal” or “patient” or “mammal,” is meant any subject, particularly a mammalian subject, for whom diagnosis, prognosis, or therapy is desired. Mammalian subjects include humans and other primates, domestic animals, farm animals, and zoo, sports, or pet animals such as dogs, cats, guinea pigs, rabbits, rats, mice, horses, donkeys, mules, burros, cattle, cows, and the like.

Overview

The present invention provides pharmaceutical compositions and methods that overcome the limitations of previously reported treatments and methods for treating and/or preventing CVD. Thus, in certain embodiments the present invention provides compositions for treating and/or preventing cardiovascular disease in hypercholesterolemic and hypertensive patients. Furthermore, the compositions and methods of the present invention can affect vascular function and endothelium dependent vasodilation, through modulation of lipoprotein formation, lipoprotein oxidation, angiotensin physiology and reduction of vascular oxidative stress thereby in an innovative integral manner reducing the development and progression of CVD. Atorvastatin or any other similar compound of the “statin” family, ramipril or any other similar compound of the “pril” family, lycopene or other effective known antioxidants and acetylsalicylic acid, which are used in combination in exemplary compositions provided by the present invention, each have different but complimentary mechanisms of action to affect vascular function.

The compositions and methods provided by the present invention can be used not only for the treatment and/or prevention of cardiovascular disease, but also for the treatment and/or prevention of any other diseases that cause and produce damage to the vascular endothelium. Additionally, the use of the compositions of the present invention is not restricted to human beings; it can be also used in any mammal, alone or in combination with any other medicines or pharmaceutically active compound or compositions designed for the treatment of cardiovascular disease symptoms.

While not wishing to be bound by any particular theory, it is believed that one of the components of certain exemplary compositions of the present invention, atorvastatin, works in the present compositions and methods by reducing cholesterol synthesis through the inhibition of hydroxy-3-methylglutaryl-coenzyme A (HMG-CoA). Inhibition of HMG-CoA is known to reduce cholesterol synthesis and improve endothelium-vasomotion in short term treatments. Furthermore, it is believed that lipid lowering treatments downregulate the angiotensin II type 1 receptor and reduce the release of free radicals. Thus, treatment with atorvastatin protects the vascular endothelium from oxidative damage and reverses the elevated blood pressure, thereby reducing the progression of atherosclerosis and development of cardiovascular disease.

It is also believed that one of the components of the compositions of the present invention, atorvastatin also has an inhibitory effect on vascular superoxide generation and increases human paraoxonase activity (a protective enzyme against LDL oxidation). This is in addition to contributing to the reduction in LDL cholesterol and consistent with enhanced nitric oxide (NO) bioactivity. Thus, both mechanisms of action for atorvastatin protect LDL from oxidation. Furthermore, atorvastatin may have anti-atherosclerotic effects independent of LDL reduction; for example, atorvastatin treatment can produce a small increase in the anti-artherogenic HDL cholesterol. Thus, atorvastatin has pleiotropic effects on the vascular endothelial architecture: inhibition of smooth muscle cell proliferation, reduction of matrix metalloproteinase expression, and stimulation of the antithrombotic system.

Similarly, while not wishing to be bound by any particular theory, it is believed that another of the components of certain exemplary compositions of the present invention, ramipril, works in the present compositions and methods to interrupt angiotensin II formation by inhibiting the angiotensin converting enzyme. Ramipril is known to reduce bradykinin degradation and diminish intracellular production of superoxide anions thus protecting LDL cholesterol from oxidation and thereby improving endothelial function. A reduction in bradykinin degradation augments NO bioactivity with activation of endothelial B2 kinin receptors and stimulation of NO synthase activity. ACE inhibition also diminishes intracellular production of superoxide anions via reduced activity of angiotensin II-dependent oxidases in the endothelium and vascular smooth muscle, thus protecting NO from oxidant degradation to biologically inert or toxic molecules. Inhibition of the production of superoxide anions also limits the oxidation of LDL, thus contributing to an increase in NO bioactivity by enhancing NO synthesis and limiting oxidative degradation of NO. Ramipril thus prevents LDL from oxidation and attenuates atherosclerosis. In this way, it is believed that ramipril promotes the diminishment of intracellular production of superoxide anions protecting LDL cholesterol from oxidation and reduces bradykinin degradation, thus improving overall vascular endothelium function and reducing hypertension.

Likewise, while not wishing to be bound by any particular theory, it is believed that one of the components of certain exemplary compositions of the present invention, acetylsalicylic acid, works to reduce the activation and aggregation of platelets by inhibiting cyclooxygenases (COX-1 and COX-2) and the formation of thromboxane. In this way, it is believed that acetylsalicylic acid reduces the release of inflammatory cytokines at the site of vascular endothelial injury, thus attenuating major vascular events.

Similarly, while not wishing to be bound by any particular theory, it is believed that one of the components of certain exemplary compositions of the present invention, lycopene, prevents atherosclerosis and CVD by two mechanisms; one of them being related to its ability to inhibit cellular 3-hydroxy-3-methylglutaryl-CoA reductase activity and expression, thus leading to decreased plasma LDL cholesterol levels. The second mechanism contributing to the atheroprotective effects of lycopene is attributed to its ability to reduce oxidative stress and oxidation of LDL. Inhibition of the oxidation of LDL contributes to an increase in NO bioactivity by enhancing NO synthesis and limiting oxidative degradation of NO. In addition, lycopene also attenuates inflammatory cytokine-stimulated endothelial cell adhesion molecule expression and monocyte-endothelial interactions. In this way, it is believed that lycopene reduces the incidence of myocardial infarction, ischemic heart disease and stroke.

Likewise, while not wishing to be bound by any particular theory, it is believed that a combination of the components of the compositions of the present invention, acetylsalicylic acid and atorvastatin, have an additional positive effect on CVD development and progression via a reduction of vascular oxidative stress. Acetylsalicylic acid is believed to inhibit the expression of the lectin-like receptor LOX-1 that is induced by oxidized low density lipoprotein in endothelial cells. This inhibition is associated with an inhibition of the expression of matrix-metalloproteinase-I. The inhibitory effect of acetylsalicylic acid in the expression of lectin-like receptor and matrix-metalloproteinases-1 improved endothelial NO bioavailability, protecting endothelial cells from vascular oxidative stress. As discussed herein, a reduction in NO bioavailability increases vascular oxidative stress thus promoting the atherosclerotic process. Statin treatment, such as with atorvastatin, is known to reduce platelet aggregation, possibly via reduction of thromboxane A2 production and cholesterol content of platelet membranes, and is known to reduce thrombogenic potential, via an effect on tissue factor. Thus, the combination of acetylsalicylic acid and atorvastatin has a synergistic effect in reducing atherothrombotic risk.

Likewise, while not wishing to be bound by any particular theory, it is believed that a combination of the components of the compositions of the present invention, lycopene and atorvastatin, have an additional positive effect on CVD development and progression via a reduction of plasma cholesterol LDL concentrations in blood and by protecting LDL from oxidation. Atorvastatin reduces cholesterol synthesis through the inhibition of hydroxy-3-methylglutaryl-coenzyme A (HMG-CoA), while lycopene in addition to inhibiting the activity of HMG-CoA also reduces its expression. Additionally, both components reduce the oxidation rate of LDL, protecting the vascular endothelium from oxidative damage, thereby reducing the progression of atherosclerosis and development of cardiovascular disease.

Hypercholesterolemia, LDL oxidation and angiotensin II degradation have been identified as some of the most important promoters of the vascular endothelial damage. Therefore, while not wishing to be bound by any particular theory, it is believed that a combination of the components of the present invention, ramipril, atorvastatin and lycopene, have an additional positive effect on endothelial function. Ramipril works in the present compositions and methods to interrupt angiotensin II formation by inhibiting the angiotensin converting enzyme, reducing intracellular production of superoxide anions thus protecting LDL cholesterol from oxidation and improving endothelial function. Atorvastatin works in the present compositions by inhibiting hydroxy-3-methylglutaryl-coenzyme A (HMG-CoA) reductase. Inhibition of HMG-CoA is known to reduce cholesterol synthesis and improve endothelium-vasomotion in short term treatments. Lycopene works in the present invention by decreasing plasma LDL cholesterol levels, by inhibiting cellular HMG-CoA reductase activity and expression, and reducing oxidative stress and oxidation of LDL. Furthermore, it is believed that lipid lowering treatments downregulate the angiotensin II type 1 receptor and reduce the release of free radicals. Thus, atorvastatin, ramipril and lycopene protect the vascular endothelium from oxidative damage and reverse the elevated blood pressure, thereby reducing the progression of atherosclerosis and development of cardiovascular disease in a complementary and synergistic manner.

Compositions

In one embodiment, the present invention provides pharmaceutical compositions useful for the treatment of cardiovascular disease and related risk factors, such as, for example, hypercholesterolemia, hypertrophy, hypertension, congestive heart failure, myocardial ischemia, ischemia reperfusion injuries in an organ, arrhythmia, and myocardial infarction, in a mammal. Exemplary pharmaceutical compositions according to this aspect of the invention comprise a therapeutically effective amount of each of at least one cholesterol lowering agent, at least one angiotensin converting enzyme inhibitor, at least one antioxidant agent and at least one antiplatelet agent. By “at least one” such agent is meant that one or more (e.g., one, two, three, four, five, etc.) of each of these agents may be present in combination in the composition of the present invention, but at least one of each of the respective classes of agents must be present in the same composition.

In certain embodiments, the invention provides pharmaceutical compositions for the treatment of cardiovascular disease in a mammal, comprising a therapeutically effective amount of at least one antiplatelet agent. Examples of antiplatelet agents suitable for use in the compositions of the invention comprise, but are not limited to, cyclooxygenase inhibitors, adenosine diphosphate (ADP) receptor inhibitors, phosphodiesterase inhibitors, glycoprotein inhibitors, and adenosine reuptake inhibitors. Other suitable antiplatelet agents that can be advantageously used in the compositions of the present invention will be familiar to one of ordinary skill. Examples of antiplatelet agents suitable for use in the present invention comprise, but are not limited to acetylsalicylic acid, warfarin, ticlopidine, clopidogrel, dypyridamole, among others. In particular compositions, the antiplatelet agent is acetylsalicylic acid. In certain such embodiments, an antiplatelet agent is present in the pharmaceutical compositions in an amount from about 20 mg to about 500 mg of an antiplatelet agent. In other embodiments, the antiplatelet agent is present in the compositions in an amount from about 35 mg to about 350 mg. In other embodiments, the antiplatelet agent is present in the compositions in an amount from about 35 mg to about 100 mg. In specific embodiments, acetylsalicylic acid is present in the compositions in an amount of about 81 mg.

In certain embodiments, the invention provides pharmaceutical compositions for the treatment of cardiovascular disease in a mammal, comprising a therapeutically effective amount of at least one angiotensin converting enzyme inhibitor. Examples of angiotensin converting enzyme inhibitors suitable for use in the compositions of the invention comprise, but are not limited to captopril, enalapril, lisinopril, benazapril, fosinopril, quinapril, ramipril, spirapril, imidapril, and moexipril. Other suitable angiotensin converting enzyme inhibitors that can be advantageously used in the compositions of the present invention will be familiar to one of ordinary skill. In particular compositions, the angiotensin converting enzyme inhibitor is ramipril.

In certain such embodiments, an angiotensin converting enzyme inhibitor is present in the pharmaceutical compositions of the invention in an amount from about 1 mg to about 80 mg of an angiotensin converting enzyme inhibitor. In other embodiments, the angiotensin converting enzyme inhibitor is present in the compositions in an amount from about 5 mg to about 60 mg. In specific embodiments, ramipril is present in the compositions in an amount of about 5 mg.

In certain embodiments, the invention provides pharmaceutical compositions for the treatment of cardiovascular disease in a mammal, comprising a therapeutically effective amount of at least one least one cholesterol lowering agent. Examples of cholesterol lowering agents suitable for use in the compositions of the invention comprise, but are not limited to, statins, fibrates, niacins and derivatives thereof, and bile acid sequestrants. Other suitable cholesterol lowering agents that can be advantageously used in the compositions of the present invention will be familiar to one of ordinary skill. In particular methods, the cholesterol lowering agent is atorvastatin. In certain such embodiments, the cholesterol lowering agent is present in the pharmaceutical compositions in an amount from about 5 mg to about 140 mg of cholesterol lowering agent. In other embodiments, the cholesterol lowering agent is present in the compositions in an amount from about 20 mg to about 80 mg. In specific embodiments, atorvastatin is present in the compositions in an amount of about 20 mg.

In certain embodiments, the invention provides pharmaceutical compositions for the treatment of cardiovascular disease in a mammal, comprising a therapeutically effective amount of at least one antioxidant agent. Examples of antioxidant agents suitable for use in the compositions of the invention comprise, but are not limited to, β-carotene, vitamin E, vitamin C, lycopene and Selenium. Other suitable antioxidant agents that can be advantageously used in the compositions of the present invention will be familiar to one of ordinary skill. In particular methods, the antioxidant agent is lycopene. In certain such embodiments, the antioxidant agent is present in the pharmaceutical compositions in an amount from about 5 mg to about 500 mg of antioxidant agent. In other embodiments, the antioxidant agent is present in the compositions in an amount from about 30 mg to about 300 mg. In specific embodiments, lycopene is present in the compositions in an amount of about 30 mg.

The compounds are combined in a single formulation, for example, as a tablet, capsule, syrup or solution. The combination of the four agents in a single oral dosage form provides the additional advantage of allowing reduced dosages of the active ingredients, increasing the compliance to the treatment and consequently a greater effect on the treatment and prevention of cardiovascular disease.

The compositions of the present invention can be administered to a patient via any suitable mode of administration, including orally, buccally, topically, transdermally, sublingually, parenterally or the like. In certain embodiments, the compositions are administered orally. Such oral administration can be accomplished via liquid or solid formulations of compositions of the invention. Methods of oral administration of pharmaceutical compositions are well within the level of skill of the ordinarily skilled artisan, and are also described hereinbelow.

Thus, in certain embodiments, the compositions of the invention may be formulated into forms for oral administration, including solid dosage forms or liquid dosage forms. In alternative embodiments, the compositions of the invention may be formulated into forms for direct administration to the mucosa, including the buccal mucosa (i.e., buccal administration) or oral mucosa under the tongue (i.e., sublingual administration). Solid dosage forms for oral administration include capsules, tablets, pills, powders, particles and granules. In such solid dosage forms, the compositions of the invention are mixed with at least one pharmaceutically acceptable excipient or carrier such as (a) fillers or extenders such as starches, lactose, sucrose, glucose, mannitol, dicalcium phosphate and microcrystalline cellulose; (b) binders such as sodium carboxymethylcellulose, alginates, gelatin, polyvinylpyrrolidone, and acacia; (c) disintegrating agents such as agar-agar, calcium carbonate, potato or tapioca starch, alginic acid, certain silicates, sodium carboxymethyl cellulose, pregelatinized starch and sodium starch glycolate; (d) lubricants such as calcium stearate, magnesium stearate, stearic acid, solid polyethylene glycols, sodium lauryl sulfate, and mixtures thereof; and/or (e) glidants such as talc, silicon dioxide and starch. In the case of capsules, tablets and pills, the dosage form may also comprise buffering agents. Solid compositions of a similar type may also be employed as fillers in soft and hard filled gelatin capsules using such excipients as lactose or milk sugar as well as high molecular weight polyethylene glycols, oils and the like. The solid dosage forms of tablets, dragees, capsules, pills, and granules can be prepared with coatings or shells such as enteric coatings and other coatings that are well known in the pharmaceutical formulating art. The solid dosage forms also may optionally contain opacifying, coloring and/or flavoring agents, and can also be formulated such that they release the active ingredient(s) only, or preferentially, in a certain part of the intestinal tract, optionally in a delayed manner (see U.S. Pat. No. 5,271,946, the disclosure of which is incorporated herein by reference in its entirety). Examples of embedding compositions which can be used include polymeric substances and waxes. The active compounds can also be in micro-encapsulated form, if appropriate, with one or more of the above-mentioned excipients.

In other embodiments, the compositions of the invention are formulated into dosage forms suitable for parenteral administration. For example, liquid dosage forms of the compositions of the present invention that are suitable for parenteral (including via injection) or oral administration include pharmaceutically acceptable emulsions, solutions, suspensions, syrups and elixirs. In addition to the active compound(s), the liquid dosage forms may contain inert diluents and/or solvents commonly used in the art. Water is the solvent of choice for the formulations of the invention; however, combinations of water with other physiologically acceptable solvents as required are also satisfactory for use. Other solvents, solubilizing agents and emulsifiers suitable for use in place of, or in addition to, water include but are not limited to saturated aliphatic mono- and polyvalent alcohols which contain 2-6 carbon atoms (including, but not limited to, ethanol, 1,2-propylene glycol, sorbitol, and glycerine), polyglycols such as polyethylene glycols, and surfactants/emulsifiers like the fatty acid esters of sorbitan, and mixtures thereof. Oils, in particular, cottonseed, peanut, or corn oils, may also be added to the compositions. The combination of the additional solvents in the aqueous solution should preferably not exceed about 15% (w/v) of the total composition. Besides inert diluents, the oral compositions can also include adjuvants such as wetting agents, emulsifying and suspending agents (e.g., microcrystalline cellulose, sodium carboxymethyl cellulose, hypromellose, carbopol and the like), surfactants, sweetening, flavoring, and perfuming agents, including those described in further detail herein below. Liquid dosage forms that provide the active ingredient(s) in suspension may comprise, in addition to the active compound(s), one or more suspending agents such as microcrystalline cellulose, magnesium aluminum silicate, bentonite, agar-agar, hypromellose, sodium carboxymethyl cellulose, carbopol/carbomer, pectin, acacia, tragacanth or their mixtures.

Suitable formulations for parenteral administration (e.g., via injection, particularly intraarticular injection) include aqueous solutions of the active compounds in water-soluble form, for example water-soluble salts and alkaline solutions. Alkaline salts can include ammonium salts prepared, for example, with Tris, choline hydroxide, bis-Tris propane, N-methylglucamine, or arginine. In addition, suspensions of the active compounds as appropriate oily injection suspensions can be administered. Suitable lipophilic solvents or vehicles include fatty oils, for example, sesame oil, or synthetic fatty acid esters, for example, ethyl oleate or triglycerides or polyethylene glycol-400 (the compounds are soluble in PEG-400). Aqueous injection suspensions can contain substances that increase the viscosity of the suspension, for example sodium carboxymethyl cellulose, sorbitol, and/or dextran. Optionally, the suspension may also contain stabilizers.

Certain compositions of the invention may further comprise one or more solubility-enhancing agents that are used to improve the solubility of the compositions used as active ingredients in the compositions of the invention. Solubility-enhancing agents that are suitable for use in the compositions of the invention include, but are not limited to, polyvinylpyrrolidone (preferably grades 25, 30, 60, or 90), poloxamer, polysorbate 80, sorbitan monooleate 80, and polyethylene glycols (molecular weights of 200 to 600). In addition to active agents, the compositions of the invention can optionally comprise one or more pharmaceutical excipients well-known in the relevant arts. Typically, such compositions are administered orally. The optimal amounts of each active agent in the composition can be determined by the clinical practioner using routine methods known to the ordinarily skilled artisan based on the guidance provided herein and in view of the information that is readily available in the art.

The compositions of the present invention may be administered as part of a pharmaceutical composition comprising the compositions of the invention and one or more suitable pharmaceutically acceptable carriers, such as one or more excipients or auxiliaries which facilitate processing of the compounds into preparations which can be used pharmaceutically. Preferably, such pharmaceutical compositions contain the amounts of the active compound(s) described herein, together with the excipient(s), particularly those compositions which can be administered orally or topically and which can be used for the preferred type of administration, such as tablets, dragees, slow release lozenges and capsules, mouth rinses and mouthwashes, gels, liquid suspensions, and also preparations which can be administered rectally, such as suppositories, as well as suitable solutions for administration by parenteral administration, e.g., via intravenous infusion, intramuscular or subcutaneous injection.

The pharmaceutical compositions of the invention may be administered to any patient who is in need of receiving them, or who experience the beneficial effects of the compositions of the invention. Foremost among such patients are mammals who are suffering from CVD or who are experiencing one or more symptoms of or precursors to CVD such as those described herein. Mammals that are suitably the subject of the treatment and/or preventive benefits provided by the compositions and methods of the present invention include humans, although the invention is not intended to be so limited. Other patients include veterinary animals (cows, sheep, pigs, horses, dogs, cats and the like).

The pharmaceutical compositions of the invention may be administered by any means that achieves their intended purpose. For example, administration may be by parenteral, subcutaneous, intravenous, intramuscular, intradermal, intraperitoneal, transdermal, buccal, sublingual, intrathecal, intracranial, ocular, or topical routes. Alternatively, or concurrently, administration may be by the oral route. The dosage administered will be dependent upon the age, health, and weight of the recipient, kind of concurrent treatment, if any, frequency of treatment, and the nature of the effect desired.

Suitable oral pharmaceutical compositions of the present invention are manufactured in a manner which is itself well-known in the art, for example, by means of conventional mixing, granulating, dragee-making, dissolving, or lyophilizing processes. Thus, solid pharmaceutical preparations for oral use can be obtained by combining a pharmaceutical composition of the invention and optionally one or more additional active pharmaceutical ingredients with one or more solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose, sucrose, fructose and the like; sugar alcohols such as mannitol, sorbitol, or xylitol and the like; cellulose preparations and/or calcium phosphates, for example tricalcium phosphate or calcium hydrogen phosphate; as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or poly(ethylene glycol). Dragee cores are provided with suitable coatings which, if desired, are resistant to gastric juices. For this purpose, concentrated saccharide solutions may be used, which may optionally contain gum arabic, talc, polyvinyl pyrrolidone, poly(ethylene glycol) and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, can be used. Dye stuffs or pigments may be added to the tablets or dragee coatings, for example, for identification or in order to characterize combinations of active ingredients or doses thereof.

Other pharmaceutical preparations which can be used orally include push-fit capsules made of gelatin, as well as soft, sealed capsules made of gelatin and a plasticizer such as glycerol or sorbitol. In certain embodiments, the push-fit capsules can comprise a pharmaceutical composition of the invention in the form of granules which may be mixed with fillers such as lactose, binders such as starches, and/or lubricants such as talc or magnesium stearate and, optionally, stabilizers. In soft capsules, one or more pharmaceutical ingredients (e.g., a pharmaceutical composition of the invention and optionally one or more additional active pharmaceutical ingredients) are preferably dissolved or suspended in suitable liquids, such as fatty oils, or liquid paraffin. In addition, stabilizers may be added.

Suitable pharmaceutical preparations which can be used rectally include, for example, suppositories, which comprise a combination a pharmaceutical composition of the invention with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, poly(ethylene glycols), or paraffin hydrocarbons.

In addition to the solid dosage forms disclosed throughout, the present invention also provides chewable oral formulations. In certain such embodiments, the formulations will comprise (or consist essentially of) an effective amount of a pharmaceutical composition of the invention along with suitable excipients that allow the formulations to be chewed by the patient. In additional embodiments, the formulations can further comprise one or more taste-masking or sweetening agents, such as those described herein. In one embodiment, sucralose is used in the chewable formulations. Additional active agents, such as those described herein, can also optionally be added to the chewable formulations. The amount of a pharmaceutical composition of the invention, other optional active agents and sweetening agents (e.g., sucralose) in the chewable formulations of the present invention are readily determinable by those of ordinary skill in the art, and include those amounts and combinations described herein. For example, the chewable formulations of the present invention comprise (or consist essentially of) a pharmaceutical composition of the invention and about 0.05% to about 0.15% sucralose. Such chewable formulations are especially useful in patient populations where compliance is an issue, such as children, the elderly, and patients who may have difficulty swallowing or using spray/inhalable formulations.

The formulations may also contain colorants to improve the appearance of the chewable formulations, especially since an attractive coloration imparted by a colorant may improve patient compliance. The relative amounts of the colorants selected will vary depending upon the particular hue of the individual colorants and the resultant color desired.

Any standard pharmaceutically acceptable excipient can be used in the chewable tablet formulations which provides adequate compression such as diluents (e.g., mannitol, xylitol, maltitol, lactitol, sorbitol, lactose, sucrose, and compressible sugars such as DiPac® (dextrinized sucrose), available from Austin Products Inc. (Holmdel, N.J.), binders, disintegrants, splitting or swelling agents (e.g., polyvinyl polypyrrolidone, croscarmellose sodium (e.g., Ac-Di-Sol available from FMC BioPolymer, Philadelphia, Pa.), starches and derivatives, cellulose and derivatives, microcrystalline celluloses, such as Avicel™ PH 101 or Avicel™ CE-15 (a microcrystalline modified with guar gum), both available from FMC BioPolymer, (Philadelphia, Pa.), lubricating agents (e.g., magnesium stearate), and flow agents (e.g., colloidal silicon dioxide, such as Cab-O-Sil M5® available from Cabot Corporation, Kokomo, Ind.).

Suitable amounts of sweetener (e.g., sucralose) used in the chewable formulations, will be familiar to, and can be readily determined by, those skilled in the art. In certain embodiments, the sweetener is present in an amount from about 0.05% to about 5.0% (e.g., about 0.05%, about 0.1%, about 0.125%, about 0.15%, about 0.2%, about 0.3%, about 0.4%, about 0.5%, about 0.6%, about 0.7%, about 0.8%, about 0.9%, about 1%, about 1.25% about 1.5%, about 1.75%, about 2%, about 2.25%, about 2.5%, about 2.75%, about 3%, about 3.25%, about 3.5%, about 3.75%, about 4%, about 4.25%, about 4.5%, about 4.75% or about 5%). Those or ordinary skill in the art will appreciate that the amount of sweetener may vary depending on the strength of the particular sweetener used and the levels approved by the regulatory authorities for use in pharmaceutical products.

Suitable cyclodextrins for use in the chewable formulations of the present invention include α, β, or γ cyclodextrins, or an alkylated or hydroxyalkylated derivatives thereof, such as heptakis (2,6-di-o-methyl)-β-cyclodextrin (DIMEB), randomly methylated β-cyclodextrin (RAMEB), and hydroxypropyl β-cyclodextrin (HPβCD). A suitable cyclodextrin is β-cyclodextrin (available from Cerestar USA, Inc., Hammond, Ind. or from Roquette America, Inc., Keokuk. IA under the trade name Kleptose™). If desired, the complex of the active substance with cyclodextrin can be prepared in advance, for example, by malaxating or granulating a pharmaceutical composition of the invention and any additional active substance(s) and the cyclodextrin in the presence of water, or by preparing an aqueous solution containing a pharmaceutical composition of the invention and any additional active substance(s) and the cyclodextrin in the desired molar ratio. Alternatively, the pharmaceutical composition of the invention and any additional active substance(s) and the cyclodextrin can be simply mixed with other excipients and adjuvants.

A typical manufacturing process for making either a single layer or bi-layer chewable tablet generally involves blending of the desired ingredients to form a uniform distribution of the pharmaceutical composition of the invention (and any other active agent(s)), excipients (e.g., colorants and flavoring agents as well as others). If desired, an inclusion complex of a pharmaceutical composition of the invention and any other active agent(s) and cyclodextrin (e.g., β-cyclodextrin) may be formed prior to blending into the mixture by malaxating a pharmaceutical composition of the invention and any other active agent(s) and cyclodextrin in the presence of water in a planetary mixer for about 20 minutes. The mixture is then dried in a drying oven. After drying, the complex is mixed with any color/flavoring blend. The blend is then compressed into a single layer or bi-layer tablet using standard methods well-known to those skilled in the art (e.g., Kilian T-100 tablet press or Courtoy 292/43 rotary bi-layer press). The colorants and flavoring agents may be added to both layers to form a uniform presentation of the tablet. Methods for preparation of chewable tablets and various components for use in the tablets can be found throughout the detailed description section and the Examples of U.S. Patent Publication No. 2003/0215503, the disclosure of which is incorporated by reference herein for all purposes. Additional chewable/orally dissolving tablets, and methods for their manufacture, are disclosed in U.S. Patent Publication No. 2004/0265372 and U.S. Pat. No. 6,270,790, the disclosures of each of which are incorporated by reference herein for all purposes.

In another embodiment, the present invention provides orally disintegrating/orodispersible tablets, such as those disclosed in U.S. Pat. No. 6,723,348, the disclosure of which is incorporated herein by reference in its entirety for all purposes. The orally disintegrating/orodispersible tablets suitably disintegrate in the buccal cavity upon contact with saliva forming an easy-to-swallow suspension. Such tablets comprise a pharmaceutical composition of the invention, and optionally, one or more additional active agents (such as those described herein), in the form of coated granules, and a mixture of excipients comprising at least one disintegrating agent, a soluble diluent agent, a lubricant and optionally a swelling agent, an antistatic (fluid flow) agent, a permeabilising agent, sweeteners, flavoring agents and colors.

In other suitable embodiments, the particles/granules of compositions of the invention (and any other optional active agents) have a particle size such that about 100% of the particles have an average size of less than about 50 μm. In suitable such embodiments, a pharmaceutical composition of the invention (and any other optional active agents) are present as coated granules.

In one embodiment, disintegrating tablets according to this aspect of the invention comprise coated granules of compositions of the invention and a mixture of excipients, the ratio of the mixture of excipients to the coated granules suitably is about 0.4:1 to about 9:1, more suitable about 1.5:1 to about 5:1, or about 2 to 3 parts by weight, the mixture of excipients suitably comprising: at least one disintegrating agent, a soluble diluent agent, a lubricant, and optionally a permeabilising agent, a swelling agent, an antistatic agent, flavoring agents and one or more coloring agents.

In suitable embodiments, the disintegrating agent is selected from the group consisting of croscarmellose, available as e.g. Ac-di-sol™, crospovidone available as e.g. Kollidon CL™, sodium starch glycolate and mixtures thereof.

According to one embodiment of the invention, the soluble diluent is a polyol having less than 13 carbon atoms and being either in the form of a directly compressible product with an average particle size of about 100 to 500 μm, or in the form of a powder with an average particle size of less than about 100 μm, this polyol suitably being selected from the group consisting of mannitol, xylitol, sorbitol and maltitol. The proportion of disintegrating agent suitably is from about 3 to about 15% by weight, e.g., about 5 to about 15% by weight, and in the case of a mixture, each disintegrating agent being present between about 1 and about 10% by weight, e.g., about 5 to about 10% by weight, and the proportion of soluble diluent agent being about 30 to about 90% by weight, e.g., about 40 to about 60% by weight, based in each case on the weight of the tablet.

Suitable lubricants for use in the disintegrating tablets include, but are not limited to, magnesium stearate, stearic acid, sodium stearyl fumarate, micronised polyoxyethyleneglycol (micronised Macrogol 6000), leukine, sodium benzoate and mixtures thereof. The amount of lubricant generally is from about 0 to about 3%, e.g., from about 1 to about 2% by weight, based on the weight of the tablet. The lubricant can be dispersed within the mixture of excipients, or according to one embodiment, sprayed over the outer surface of the tablet. Thus, according to one embodiment of the disintegrating tablets of the invention, the lubricant is in powder form and is, at least in part, disposed on the surface of the tablets.

The permeabilising agent allows the creation of a hydrophilic network which facilitates the penetration of saliva and hence assists the disintegration of the tablet. Suitable permeabilising agent include, but are not limited to, silica with a high affinity for aqueous solvents, such as colloidal silica (Aerosil™), precipitated silica (Syloid™ FP 244), maltodextrins, β-cyclodextrins and mixtures thereof. The amount of permeabilising agent suitably is between about 0 and about 5%, e.g., from about 0.5 to about 2% by weight, based on the weight of the tablet.

A swelling agent can be incorporated in the mixture of excipients. Suitable swelling agents include, but are not limited to, starch, modified starch or microcrystalline cellulose.

An antistatic agent can also be incorporated as a flow aid. Suitable antistatic agents include, but are not limited to, micronised or non-micronised talc, fumed silica (Aerosil™ R972), colloidal silica (Aerosil™ 200), precipitated silica (Syloid™ FP 244), and mixtures thereof.

According to one such embodiment of the invention, the granules of the compositions of the invention are characterized in that the granules are coated and comprise microcrystals of composition(s) of the invention, at least one binder, and optionally a diluent agent, an antistatic agent, and a coloring agent. Furthermore, the granulation excipients can also include disintegrating agents and/or surfactants.

Suitable binders include, but are not limited to, cellulosic polymers, such as ethylcellulose, hydroxypropylcellulose and hydroxypropylmethyl cellulose, acrylic polymers, such as insoluble acrylate ammoniomethacrylate copolymer, polyacrylate or polymethacrylic copolymer, povidones, copovidones, polyvinylalcohols, alginic acid, sodium alginate, starch, pregelatinized starch, sucrose and its derivatives, guar gum, poly(ethylene glycol), for example an acrylic polymer, such as Eudragit™ E100, and mixtures thereof.

Optionally, in order to enhance the granulation of the composition(s) of the invention a diluent agent can be used. Suitable diluent agents include, but are not limited to, microcrystalline cellulose, sucrose, dicalcium phosphate, starches, lactose and polyols of less than 13 carbon atoms, such as mannitol, xylitol, sorbitol, maltitol, pharmaceutically acceptable amino acids, such as glycin, and their mixtures.

In one embodiment, a granule of one or more of the composition(s) of the invention, can be in the form of a core of granulated microcrystals of one or more compositions of the invention, coated with at least one layer comprising a composition of the invention. Such a coated core is characterized in that the core and the layer comprise each from 70% to 95%, preferably 80% to 95% by weight of one or more compositions of the invention, the balance to 100% being formed with at least one binder and optionally sucralose, and that the coated core is suitably a sphere. See e.g., French patent application FR 00 14803, the disclosure of which is incorporated by reference herein.

The granules can also be coated with a coating composition comprising at least one coating polymer selected from the group consisting of cellulosic polymers, acrylic polymers and their mixtures. Among the cellulosic polymers, ethylcellulose, hydroxypropylcellulose (HPC) and hydroxypropylmethylcellulose (HPMC), can be used. Among the acrylic polymers, insoluble acrylate ammonio-methacrylate copolymer (Eudragit™ RL100 or RS100 or Eudragit™ RL30D or RS30D), polyacrylate (Eudragit™ NE30D), or methacrylic copolymers (e.g., Eudragit™ L100-55 Eudragit™ L30D, Eudragit™ E100 and Eudragit™ EPO) can be used, alone, in combination, or in admixture with pH-dependent polymers. Eudragit™ E100 or a mixture of Eudragit™ EPO and Eudragit™ NE30D are suitably used. In one embodiment, the binder and the coating polymer are the same polymer.

Optionally, permeabilising agents, plasticizers, soluble agents, disintegrating agents and surfactants, can be added as coating additives. Suitable plasticizers include, but are not limited to, triacetine, triethylacetate, triethylcitrate (Eudraflex™), ethylphthalate, or mixtures thereof. The plasticizer is used in proportions of at most about 30%, preferably 10% by weight of the coating polymers. Suitable soluble agents include polyols having less than 13 carbon atoms. Surfactants may be an anionic, nonionic, cationic, zwitterionic or amphoteric surfactant. Suitable disintegrating agents include, but are not limited to, croscarmellose, available as e.g. Ac-di-sol™, crospovidone available as e.g. Kollidon CL™, and mixtures thereof.

Suitably, the coated granules according to the present invention have a particle size distribution between about 150 μm and about 500 μm, more suitably between about 150 μm and about 425 μM, such that at least 50%, more suitably at least 70% of the granules have a particle size ranging between about 150 and about 425 μm, and less than 15% of the granules have a particle size less than about 150 μm.

In one embodiment, the coated granules according to the invention comprise: from about 10% to about 95%, preferably about 40 to about 75% of granules of a composition of the invention and optionally one or more optional additional active agents, such as those disclosed herein, sucralose from about 0.05% to about 5%, from about 5 to about 90%, suitably about 10 to about 70% and even more suitably from about 25 to about 55% of a coating polymer, such as Eudragit™ E100, the percentages being expressed by weight relative to the weight of the granules of a composition of the invention, from about 0 to about 10% of a permeabilising agent, such as colloidal silica, the percentages being expressed by weight relative to the weight of the coating polymer.

In another embodiment, the present invention provides solid, effervescent, rapidly dissolving dosage forms, such as those disclosed in U.S. Pat. No. 6,245,353, the disclosure of which is incorporated herein by reference in its entirety. The effervescent, rapidly dissolving dosage forms suitably comprise one or more pharmaceutical compositions of the present invention. Such effervescent dosage forms can further comprise (a) an effervescent base comprising at least one of (i) at least one of (1) an organic edible acid and (2) a salt thereof, (ii) at least one of an alkali metal and an alkaline earth metal carbonate and bicarbonate. In certain other embodiments, the effervescent dosage form of the present invention can further comprise a pharmaceutically acceptable auxiliary ingredient.

In use of such effervescent dosage forms of the present invention, a solution or suspension of a composition of the invention is formed by adding water to the soluble or dispersible effervescent tablets or soluble granules, with evolution of CO₂ gas. The resulting effervescent solution or suspension can be ingested very easily, even by patients who have difficulties swallowing. The rapidly disintegrating tablet can also be administered so that it directly disintegrates in the mouth. A rapid release of the active ingredients or compositions is of particular importance here, to ensure a rapid onset of action.

Effervescent agents capable of releasing CO₂, which can be used in the present invention, include alkali metal carbonates or alkali metal bicarbonates, such as sodium carbonate or sodium bicarbonate. Agents for inducing CO₂ release which are suitably employed are edible organic acids, or their acidic salts, which are present in solid form and which can be formulated with the one or more compositions of the invention and the other auxiliary ingredients (as well as any other active agents) to provide granules or tablets, without premature evolution of CO₂. Edible organic acids which can be so used include for example, tartaric acid, malic acid, fumaric acid, adipic acid, succinic acid, ascorbic acid, maleic acid or citric acid. Pharmaceutically acceptable acidic salts include, for example, salts of polybasic acids which are present in solid form and in which at least one acid function is still present, such as sodium dihydrogen or disodium hydrogen phosphate or monosodium or disodium citrate.

In one such embodiment, the present invention thus provides effervescent formulations of one or more compositions of the invention including the formulations and compositions described herein, having an effervescent base comprising (a) a mixture of calcium carbonate with an organic edible acid; (b) a mixture of calcium carbonate, sodium carbonate, sodium bicarbonate and an organic edible acid; or (c) a mixture of sodium bicarbonates, sodium carbonate and an organic edible acid.

Exemplary soluble or dispersible effervescent tablets suitably comprise one or more compositions of the invention, a suitable amount of an effervescent base, and excipients. The effervescent base suitably comprises from about 100 mg to about 500 mg calcium ions as, for example, calcium carbonate, and from about 20 mg to about 1500 mg citric acid and/or its salts. In another embodiment, the effervescent base comprises from about 50 mg to about 2000 mg sodium bicarbonate, from about 20 mg to about 200 mg of sodium carbonate and from about 20 mg to about 1500 mg citric acid and/or from about 20 mg to about 500 mg tartaric acid. An additional suitable composition of the effervescent base comprises from about 50 mg to about 500 mg sodium bicarbonate, from about 20 mg to about 100 mg sodium carbonate, and from about 50 mg to about 750 mg calcium carbonate and from about 100 mg to about 1500 mg of citric acid.

The soluble/dispersible tablets can be prepared by known processes for preparing effervescent bases, such as those disclosed in U.S. Pat. No. 6,245,353, the disclosure of which is incorporated herein by reference in its entirety.

Another embodiment of the present invention provides a physiologically acceptable film that is particularly well-adapted to dissolve in the oral cavity of a warm-blooded animal including humans, and adhere to the mucosa of the oral cavity, to allow delivery of a pharmaceutical composition of the invention. Such physiologically acceptable films suitable for use in accordance with this aspect of the present invention are disclosed in U.S. Patent Application No. 2004/0247648, the disclosure of which is incorporated herein by reference in its entirety.

In one such embodiment of the present invention, an orally dissolving/consumable film comprises a modified starch, a pharmaceutical composition of the invention and optionally at least one water soluble polymer. The amount of a pharmaceutical composition of the invention present in such formulations is readily determinable by those of ordinary skill in the art, and include those amounts and combinations described herein.

The consumable films according to this aspect of the present invention may comprise one or more of the following ingredients: water, antimicrobial agents, additional film forming agents or water soluble polymers, plasticizing agents, flavorings, sulfur precipitating agents, saliva stimulating agents, cooling agents, surfactants, stabilizing agents, emulsifying agents, thickening agents, binding agents, coloring agents, triglycerides, poly(ethylene)oxides, propylene glycols, or sweeteners, fragrances, preservatives and the like, as described in U.S. Pat. No. 6,596,298, the disclosure of which is incorporated by reference herein in its entirety.

In one such embodiment, the consumable films of the present invention include a modified starch. The modified starches used in accordance with the present invention can be prepared by mechanically, chemically or thermally modifying unmodified starches. For example, modified starches may be prepared by chemically treating starches to produce, for example, acid treatment starches, enzyme treatment starches, oxidized starches, cross-bonding starches, and other starch derivatives. Starches suitable for modification to produce modified starches may be obtained from natural products such as corn, potatoes, tapioca as well as genetically modified forms of the same such as high amylose and waxy corn as well as sorghum varieties.

Examples of modified starches for use in the practice of the present invention include, but are not limited to, modified corn starches, modified tapioca starches, acid and enzyme hydrolyzed corn and/or potato starches, hypochlorite-oxidized starches, acid-thinned starches, ethylated starches, cross-bonded starches, hydroxypropylated tapioca starches, hydroxypropylated corn starches, pregelatinized modified starches, and the like. Preferred modified starches are selected from pregelatinized modified corn starches and pregelatinized modified tapioca starches.

Representative examples of commercially available modified starches useful in the present invention include PURE-COTET™ modified starches such as PURE-COTET™ B793 (a pregelatinized modified corn starch) and PURE-COTET™ B795 (a pregelatinized modified corn starch), for example, available from Grain Processing Corporation, 1600 Oregon Street, Muscatine, Iowa 52761-1494 USA.

In one such embodiment of the present invention, the modified starch is present in amounts ranging from about 1% to about 90% by weight, in another embodiment about 10% to about 90% by weight, and in yet another embodiment from about 35% to about 80% by weight of the film.

Modified starch may be included in the film alone or optionally in combination with an additional water soluble film forming polymers such as those selected from, for example, pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, poly(ethylene glycol), tragacanth gum, guar gum, acacia gum, arabic gum, polyacrylic acid, methylmethacrylate copolymers, carboxyvinyl polymers, amylose, high amylose starch, hydroxypropylated high amylose starch, pectin, dextrin, chitin, chitosan, levan, elsinan, collagen, gelatin, zein, gluten, soy protein isolate, whey protein isolate, casein and combinations thereof. A preferred water soluble polymer is pullulan. The amount of the water soluble polymer typically is up to about 99% by weight, suitably up to about 80% by weight, more suitably up to about 50% by weight, and most suitably up to about 40% by weight of the film.

Suitable formulations for oral and/or parenteral administration include aqueous solutions of one or more pharmaceutical compositions of the invention, in water-soluble form, for example, water-soluble salts and alkaline solutions. In addition, suspensions of the active ingredient(s) as appropriate oily injection suspensions may be administered. Suitable lipophilic solvents or vehicles include fatty oils, for example, sesame oil, or synthetic fatty acid esters, for example, ethyl oleate or triglycerides or poly(ethylene glycol)-400. Aqueous injection suspensions may optionally also comprise substances which increase the viscosity of the suspension including, for example, sodium carboxymethyl cellulose, sorbitol, and/or dextran. Optionally, the suspension may also contain one or more stabilizers, one or more preservatives (e.g., sodium edetate, benzalkonium chloride, and the like), and/or other components commonly used in formulating pharmaceutical compositions.

Suitable topical pharmaceutical compositions of the invention are formulated preferably as oils, creams, lotions, ointments and the like by choice of appropriate carriers. Exemplary compositions according to this aspect of the invention therefore comprise one or more pharmaceutical compositions of the invention, and one or more carriers suitable for use in preparing such pharmaceutical compositions for topical administration. Suitable such carriers include vegetable or mineral oils, white petrolatum (white soft paraffin), branched chain fats or oils, animal fats and high molecular weight alcohol (greater than C12). The preferred carriers are those in which the active pharmaceutical ingredient(s) are soluble. Emulsifiers, stabilizers, humectants and antioxidants may also be included, as well as agents imparting color or fragrance, if desired. Additionally, one or more transdermal penetration enhancers can be employed in these topical formulations. Non-limiting examples of suitable such enhancers can be found in U.S. Pat. Nos. 3,989,816 and 4,444,762, which are incorporated be reference herein in their relevant parts.

Creams are preferably formulated from a mixture of mineral oil, self-emulsifying beeswax and water in which mixture the active ingredient, dissolved in a small amount of an oil such as almond oil, is admixed. A typical example of such a cream is one which includes about 40 parts water, about 20 parts beeswax, about 40 parts mineral oil and about 1 part almond oil.

Ointments may be formulated by mixing a solution of one or more of compositions of the present invention in a vegetable oil such as almond oil with warm soft paraffin and allowing the mixture to cool. A typical example of such an ointment is one which includes about 30% almond oil and about 70% white soft paraffin by weight.

Lotions may be conveniently prepared by dissolving one or more of compositions of the present inventions in a suitable high molecular weight alcohol such as propylene glycol or poly(ethylene glycol).

Liquid dosage forms for oral administration include pharmaceutically acceptable emulsions, solutions, suspensions, syrups and elixirs. In addition to one or more pharmaceutical compositions of the invention, liquid dosage forms may contain inert diluents and/or solvents commonly used in the art. Water is the solvent of choice for the formulations of the invention; however, combinations of water with other physiologically acceptable solvents as required are also satisfactory for use. Other solvents, solubilizing agents and emulsifiers suitable for use in place of, or in addition to, water include but are not limited to saturated aliphatic mono- and polyvalent alcohols which contain 2-6 carbon atoms (including, but not limited to, ethanol, 1,2-propylene glycol, sorbitol, and glycerine), polyglycols such as poly(ethylene glycols), and surfactants/emulsifiers like the fatty acid esters of sorbitan, and mixtures thereof. Oils, in particular, cottonseed, peanut, or corn oils, may also be added to the compositions. The combination of the additional solvents in the aqueous solution should preferably not exceed about 15% (w/v) of the total composition. Besides inert diluents, the oral compositions can also include adjuvants such as wetting agents, emulsifying and suspending agents (e.g., microcrystalline cellulose, sodium carboxymethyl cellulose, hypromellose, carbopol and the like), surfactants, sweetening, flavoring, and perfuming agents, including those described in further detail herein below. Liquid dosage forms that provide the active ingredients or compositions of the invention in suspension may comprise, in addition to a pharmaceutical composition of the invention, one or more suspending agents such as microcrystalline cellulose, magnesium aluminum silicate, bentonite, agar-agar, hypromellose, sodium carboxymethyl cellulose, carbopol/carbomer, pectin, acacia, tragacanth or their mixtures.

Certain such liquid compositions of the invention may further comprise one or more preservatives and/or one or more stabilizers. Preservatives that are suitable for use in the compositions of the invention include, but are not limited to, edetic acid and their alkali salts such as disodium EDTA (also referred to as “disodium edetate” or “the disodium salt of edetic acid”) and calcium EDTA (also referred to as “calcium edetate”), benzyl alcohol, methylparaben, propylparaben, butylparaben, chlorobutanol, phenylethyl alcohol, benzalkonium chloride, thimerosal, propylene glycol, sorbic acid, and benzoic acid derivatives. The preservatives should be used at a concentration of from about 0.001% to about 0.5% (w/v) in the final composition. The combination of benzalkonium chloride, used at a concentration of from about 0.001% to about 0.5% or preferably from about 0.005% to about 0.1% (w/v), and edetic acid (as a disodium salt), used at a concentration of from about 0.005% to about 0.1% (w/v), are suitable preservative/stabilizer combination used in the compositions of the present invention.

Certain compositions of the invention may further comprise one or more solubility-enhancing agents that are used to improve the solubility of the pharmaceutical compositions of the invention or of the active ingredients contained therein. Solubility-enhancing agents that are suitable for use in the compositions of the invention include, but are not limited to, polyvinylpyrrolidone (preferably grades 25, 30, 60, or 90), poloxamer, polysorbate 80, sorbitan monooleate 80, and poly(ethylene glycols) (molecular weights of 200 to 600).

Certain compositions of the invention may further comprise one or more agents that are used to render the composition isotonic, particularly in those compositions in which water is used as a solvent. Such agents are particularly useful in compositions formulated for nasal or ocular application, since they adjust the osmotic pressure of the formulations to the same osmotic pressure as nasal or ocular secretions. Agents that are suitable for such a use in the compositions of the invention include, but are not limited to, sodium chloride, sorbitol, propylene glycol, dextrose, sucrose, and glycerine, and other isotonicity agents that are known in the art (see, e.g., Reich et al., “Chapter 18: Tonicity, Osmoticity, Osmolality and Osmolarity,” in: Remington: The Science and Practice of Pharmacy, 20^{th} Edition, Lippincott Williams and Wilkins, Philadelphia, Pa. (2000)).

It is frequently desirable that the compositions of the present invention that are to be administered in liquid form (including orally applied formulations) have a pH of about 4.5 to about 7.4, and preferably have a pH of about 5.5 to 7.1, for physiological reasons. Accordingly, in additional embodiments, the compositions of the invention may further comprise one or more buffering agents or combinations thereof, that are used to adjust and/or maintain the compositions into the desired pH range. Adjustment of pH or buffering agents that are suitable for use in the compositions of the invention include, but are not limited to, citric acid, sodium citrate, sodium phosphate (dibasic, heptahydrate form), and boric acid or equivalent conventional buffers, or combinations thereof. The appropriate amounts of buffers and buffering agents, or combinations thereof, that are to be used in the compositions of the invention are readily determined by those of ordinary skill without undue experimentation, particularly in view of the guidance contained herein and in standard formularies such as the United States Pharmacopoeia, Remington: The Science and Practice of Pharmacy, and the like, the disclosures of which are incorporated herein by reference in their entireties.

In certain embodiments, the liquid formulations of the invention, particularly those that are to be administered orally further comprise one or more taste-masking agents, one or more flavoring agents, and/or one or more sweetening agents, or a combination of such agents. Non-limiting examples of such substances include sucralose (about 0.001 to about 1%), sucrose (about 0.5 to about 10%), saccharin (including the salt forms: sodium, calcium, etc.) (about 0.01 to about 2%), fructose (about 0.5 to about 10%), dextrose (about 0.5 to about 10%), corn syrup (about 0.5 to about 10%), aspartame (about 0.01 to about 2%), acesulfame-K (about 0.01 to about 2%), xylitol (about 0.1 to about 10%), sorbitol (about 0.1 to about 10%), erythritol (about 0.1 to about 10%), ammonium glycyrrhizinate (about 0.01 to about 4%), thaumatin (TalinTh) (about 0.01 to about 2%), neotame (about 0.01 to about 2%) mannitol (about 0.5 to about 5%), menthol (about 0.01 to about 0.5%), eucalyptus oil (about 0.01 to about 0.5%), camphor (about 0.01 to about 0.5%), natural and or artificial flavors such as Artificial Custard Cream Flavor #36184 from International Flavors and Fragrances, Inc. (New York, N.Y.) (about 0.01 to about 1.0%), and the like. Sucralose, an intense sweetener marketed for food and beverage use as SPLENDA® by McNeil Nutritionals LLP (Fort Washington, Pa.), is especially effective as a sweetening and taste-masking agent in the compositions of the present invention, particularly when used at concentrations of from about 0.001% to about 1%, preferably at concentrations of from about 0.01% to about 0.5%, and more preferably at concentrations of from about 0.02% to about 0.2%, and most preferably from about 0.05% to about 0.15% (e.g., about 0.05%, about 0.06%, about 0.07%, about 0.08%, about 0.09%, about 0.10%, about 0.11%, about 0.12%, about 0.13%, about 0.14%, or about 0.15%), of the total composition. Sucralose has been shown to be useful as a taste modifying agent in oral delivery of certain pharmaceutical compositions, for example in sore throat spray products (see U.S. Pat. No. 6,319,513), oral suspensions (see U.S. Pat. Nos. 5,658,919 and 5,621,005), solid dosage forms (see U.S. Pat. No. 6,149,941), quick melt dosage forms (see U.S. Pat. No. 6,165,512) and mucosal delivery (see U.S. Pat. No. 6,552,024). Additional such compositions of the invention may comprise one or more additional taste-masking or flavoring agents, for example menthol at a concentration of from about 0.01% to about 1%, preferably at a concentration of from about 0.05% to about 0.1%.

### Methods of Use

In additional embodiments of the invention, the invention provides methods of treating mammals afflicted with certain diseases, particularly with cardiovascular disease and other related disorders described elsewhere herein and that will be familiar to the ordinarily skilled artisan, using the compositions of the present invention. In related embodiments, the invention provides such methods of treatment or prevention by administering to said mammal a cardiovascular disease-treating or cardiovascular disease-preventing amount of a composition comprising ramipril, atorvastatin, an antiplatelet agent such as acetylsalicylic acid, and lycopene.

In related embodiments, the invention provides methods of reducing or preventing the progression of cardiovascular disease to a more advanced stage of CVD in a patient, comprising administering to a patient suffering from CVD, a therapeutically effective amount of one or more of the compositions of the present invention. Certain such methods of the invention comprise administering to the patient one or more compositions of the invention that are described herein, and one or more additional active agents.

According to certain such methods of the invention, one or more compositions of the present invention are administered to a patient, such as a patient suffering from or predisposed to cardiovascular disease, via any suitable mode of administration as described elsewhere herein.

In particular such methods, the compositions are administered to the mammal via oral administration. Methods of oral administration can be accomplished via liquid or solid form, and particularly in solid form such as in tablet or capsule form, using approaches and mechanisms described elsewhere herein and others that will be familiar to the ordinarily skilled artisan.

Suitable dosages (e.g., amounts, volumes, etc.) of the compositions of the invention will be apparent from the description herein, including the Examples below. Thus in one embodiment, the invention provides a pharmaceutical composition for the treatment of cardiovascular diseases, including the underlying causes, but not limited to hypercholesterolemia and hypertension, in a mammal. Exemplary pharmaceutical compositions for use in methods according to this aspect of the invention comprise one or more cholesterol lowering agents, one or more angiotensin converting enzyme inhibitors, and one or more antiplatelet agents. In certain such embodiments, the methods of the invention, the pharmaceutical composition for the treatment of cardiovascular disease in a mammal comprises ramipril, atorvastatin, at least one antiplatelet agent such acetylsalicylic acid, and lycopene. Suitable amounts of each active ingredient present in the compositions that are advantageously used in this aspect of the invention will be apparent from the description herein, and from the Examples herein.

In particular such methods, the compositions of the invention are administered to the patient in a single dosage comprising a therapeutically effective amount of each of one or more cholesterol lowering agents, one or more angiotensin converting enzyme inhibitors, and one or more antiplatelet agents, and optionally one or more additional active ingredients. Suitable compositions for use in exemplary such methods of the invention include those compositions described herein comprising ramipril, atorvastatin, acetylsalicylic acid, and lycopene each in a therapeutically effective (i.e., CVD-treating or CVD-preventing amount).

In particular such methods, the compositions of the invention are administered to the patient in a single, daily dosage form, once per day. In alternative such methods, the compositions are administered to the patient two or more (i.e., two, three, four or more) times per day, or as needed according to the particular treatment regiment designed by the patient's physician.

The amount of the compositions of the invention administered each time throughout the treatment period can be the same; alternatively, the amount administered each time during the treatment period can vary (e.g., the amount administered at a given time can be more or less than the amount administered previously). For example, doses given during maintenance therapy may be lower than those administered during the acute phase of treatment. Appropriate dosing schedules depending on the specific circumstances will be apparent to persons of ordinary skill in the art.

It will be readily apparent to one of ordinary skill in the relevant arts that other suitable modifications and adaptations to the methods and applications described herein are obvious and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Example 1

### Analysis and Strategy for Development of a Formulation Comprising Atorvastatin, Ramipril, Acetylsalicylic Acid and Lycopene

As a strategy to reduce cardiovascular disease, we determined the effect of the combination of drugs and their doses, for use in a single daily pill to achieve a large effect in preventing cardiovascular disease with minimal adverse effects. The strategy was directed to developing a formulation to reduce four cardiovascular risk factors (low density lipoprotein cholesterol (LDL-c), LDL-c oxidation, blood pressure and platelet function).

We quantified the efficacy of the proposed formulation by reviewing published meta-analysis of randomized trials related to the effect of different drugs.

A polypill composition that meets the objective of the present invention was identified that comprises 20 mg of atorvastatin, 5 mg of ramipril, 81 mg of acetylsalicylic acid and 30 mg of lycopene.

Table 1 shows the expected proportion of people who would avoid an ischaemic heart disease by taking a polypill capsule comprising the identified formulation.

TABLE 1
Expected value of the effect of the polypill capsule on the risk of
ischaemic heart disease

| | | | % | |
|---|---|---|---|---|
| | | | reduction | |
| | | | in | Relative |
| | | | risk of | Risk of |
| | | Reduction in | Ischaemic | Ischaemic |
| | | concen- | heart | heart |
| Risk Factor | Agent | tration | disease | disease |
| LDL-cholesterol | Atorvastatin | 70 mg/dL | 61 | 0.39 |
| LDL-cholesterol | Lycopene | 14-20 mg/dL | 20 | 0.80 |
| Platelet | Acetylsalicylic | Not | 32 | 0.68 |
| Function | Acid | quantified | | |
| Blood Pressure | Ramipril | 6 mmHg | 25 | 0.75 |
| | | diastolic | | |
| Combined effect | All | | 84 | 0.16 |

Several clinical trials have shown that a reduction in LDL-cholesterol of about 70 mg/dL produces a reduction in the risk of ischaemic heart disease of about 61%. Based on the formulation according to the present invention, use of atorvastatin (20 mg) can reduce LDL-cholesterol by an average of 70 mg/dL, thereby reducing the incidence of ischaemic heart attack by an estimated 61%. A further reduction of LDL-cholesterol may be obtained from the addition of lycopene which can reduce LDL-cholesterol by about 14-20 mg/dL, therefore reducing the risk of ischaemic heart attack by an estimated additional 20%.

The blood pressure reduction of 11 mmHg diastolic, decreases the incidence of ischaemic hearth disease by about 46%. Ramipril included in the formulation of the present invention may reduce diastolic blood pressure by about 6 mmHg, which can be reflected as an estimated 25% reduction in ischaemic heart disease.

As with statins, acetylsalicylic acid may have a larger preventive effect in people with occlusive vascular disease. It has been shown that low dose of acetylsalicylic acid (75-125 mg/d) may reduce ischaemic heart disease by about 32%. Therefore, the formulation of the present invention contains the minimum effective dose of acetylsalicylic acid to produce this heart protective effect.

Merging all these four risk factors together could reduce the risk of ischaemic heart disease by about 84%. This reduction is obtained from the product of the relative risk estimates relating to intervention for each risk factor, which is the complement of the proportion of events prevented; thus a prevention of 61% is equivalent to a relative risk of 0.39.

Therefore the relative risk of an ischaemic heart disease with the intervention of the formulation of the present invention will be 0.39, 0.80, 0.68 and 0.75, the product of which yields a combined relative risk of 0.16 or an 84% preventive effect. The use of a composition according to the present invention constitutes a treatment which is more effective, has less adverse symptoms and has greater compliance.

### Example 2

### Stability and Compatibility Testing of Microtablets

The compatibility between each active ingredient (atorvastatin, ramipril, acetylsalicylic acid and lycopene) and different excipient(s) was tested. Each active ingredient and excipient(s) were mixed together. The particle size distribution and flow properties of the blended active ingredient and excipient(s) was assessed. Following mixing, the resulting powder was used for preparation of a microtablet. Microtablets were formed using direct compression, dry granulation, wet granulation and/or combinations or modifications thereof. The microtablets of each active ingredient had a diameter of about 3 mm or less. Additional microtablets were prepared that had a diameter of less than 2.5 mm. The stability and release characteristics of each active ingredient microtablet were assessed. Each active ingredient microtablet has different properties from the other active ingredient microtablets. Excipient(s) that were found to be compatible with each active ingredient were utilized in the microtablet preparation described in Example 3.

### Example 3

### Polypill Capsule Formulation Comprising Atorvastatin, Acetylsalicylic Acid and Lycopene

A polypill capsule containing 20 mg of atorvastatin, 5 mg of ramipril, 81 mg of acetylsalicylic acid and 30 mg of lycopene, was prepared as follows (Table 2).

TABLE 2
Exemplary capsule formulation comprising atorvastatin, ramipril,
acetylsalicylic acid and lycopene.

| | Amount per polypill | mg per polypill capsule, |
|---|---|---|
| Active Ingredient | capsule (mg) range | exemplary formulation |
| Atorvastatin | 5-140 | 20 |
| Ramipril | 1-80 | 5 |
| Acetylsalicylic acid | 20-500 | 81 |
| Lycopene | 5-500 | 30 |

### Methods

Mix Process

Atorvastatin was passed through a mesh screen and collected in a clean polyethylene container. Atorvastatin was then mixed with the excipient(s) that resulted in maximum stability and desired release characteristics of the atorvastatin microtablet, as identified in Example 2.

Ramipril was passed through a mesh screen and collected in a clean polyethylene container. Ramipril was then mixed with the excipient(s) that resulted in maximum stability and desired release characteristics of the ramipril microtablet, as identified in Example 2.

Acetylsalicylic acid was passed through a mesh screen and collected in a clean polyethylene container. Acetylsalicylic acid was then mixed with the excipient(s) that resulted in maximum stability and desired release characteristics of the acetylsalicylic acid microtablet, as identified in Example 2.

Lycopene was passed through a mesh screen and collected in a clean polyethylene container. Lycopene was then mixed with the excipient(s) that resulted in maximum stability and desired release characteristics of the lycopene microtablet, as identified in Example 2.

Tableting

Following mixing, the resulting powder of each active ingredient/excipient blend was individually compressed in a tablet press machine to form an active ingredient microtablet with a diameter of less than 2.5 mm.

Polypill Capsule Formation

Equivalent amounts of atorvastatin, ramipril, acetylsalicylic acid and lycopene microtablets were mixed together and encapsulated in suitable size hard gelatin capsules to yield the final product.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

What is claimed is:

1. A pharmaceutical composition for the treatment of cardiovascular disease in a mammal, comprising a therapeutically effective amount of each of at least one cholesterol lowering agent, at least one angiotensin converting enzyme inhibitor, at least one antioxidant agent and at least one antiplatelet agent.

2. The pharmaceutical composition of claim 1, wherein said cholesterol lowering agent is atorvastatin.

3. The pharmaceutical composition of claim 2, wherein said composition comprises from about 5 mg to about 140 mg of atorvastatin, preferably from about 20 mg to about 80 mg of atorvastatin.

4. The pharmaceutical composition of claim 2, wherein said composition comprises about 20 mg of atorvastatin.

5. The pharmaceutical composition of any one of claims 1-4, wherein said angiotensin converting enzyme inhibitor is ramipril.

6. The pharmaceutical composition of claim 5, wherein said composition comprises from about 1 mg to about 80 mg of ramipril, preferably from about 5 mg to about 60 mg of ramipril.

7. The pharmaceutical composition of claim 5, wherein said composition comprises about 5 mg of ramipril.

8. The pharmaceutical composition of any one of claims 1-7, wherein said antiplatelet agent is acetylsalicylic acid.

9. The pharmaceutical composition of claim 8, wherein said composition comprises from about 20 mg to about 500 mg of acetylsalicylic acid, preferably from about 35 mg to about 350 mg of acetylsalicylic acid, more preferably about 35 mg to about 100 mg of acetylsalicylic acid.

10. The pharmaceutical composition of claim 8, wherein said composition comprises about 81 mg of acetylsalicylic acid.

11. The pharmaceutical composition of any one of claims 1-10, wherein said antioxidant agent is lycopene.

12. The pharmaceutical composition of claim 11, wherein said composition comprises from about 5 mg to about 500 mg of lycopene, preferably from about 30 mg to about 100 mg of lycopene.

13. The pharmaceutical composition of claim 11, wherein said composition comprises about 30 mg of lycopene.

14. The pharmaceutical composition of any one of claims 1-13, wherein such composition comprises atorvastatin, ramipril, lycopene and an antiplatelet agent.

15. The pharmaceutical composition of claim 14, wherein said composition is administered in a single, once daily dose.

16. The pharmaceutical composition of claim 14, wherein said composition is administered as a tablet, capsule, syrup or solution.

17. The pharmaceutical composition of claim 14, wherein said atorvastatin, said ramipril, said lycopene and said antiplatelet agent are each in the form of a microtablet.

18. The pharmaceutical composition of claim 16, wherein said microtablets of atorvastatin, ramipril, lycopene and antiplatelet agent are intermixed and encapsulated within a hard gelatin capsule.

19. Use of a composition of any one of claims 1-18 to treat or prevent cardiovascular disease.

20. A kit for use in treating or preventing cardiovascular disease, comprising a pharmaceutical composition of any one of claims 1-18 and a label containing instructions to administer the pharmaceutical composition once per day, twice per day, three times per day, or four times per day to a patient in need thereof.

21. A method of treating or preventing cardiovascular disease in a subject, comprising administering to said subject in need of such treatment a pharmaceutical composition of any one of claims 1-18.
